(19) United States
(12) Patent Application Publication
(10) Pub. No.: US 2018/0321206 A1
(43) Pub. Date: Nov. 8, 2018

(54) MODULAR FLUID SENSING SYSTEM

(71) Applicant: KONINKLIJKE PHILIPS N.V., EINDHOVEN (NL)

(72) Inventors: SHUANG CHEN, EINDHOVEN (NL); DECLAN PATRICK KELLY, EINDHOVEN (NL); CORNELIS REINDER RONDA, EINDHOVEN (NL); PASCAL DE GRAAF, EINDHOVEN (NL); NICO MARIS ADRIAAN DE WILD, EINDHOVEN (NL); MARCEL MULDER, EINDHOVEN (NL); JEAN-PAUL JACOBS, EINDHOVEN (NL)

(21) Appl. No.: 15/773,206

(22) PCT Filed: Nov. 3, 2016
(86) PCT No.: PCT/EP2016/076578

§ 371 Date: May 3, 2018

(30) Foreign Application Priority Data
Nov. 6, 2015 (CN) PCT/CN2015/093978
Feb. 25, 2016 (EP) 16157294.6

(51) Int. Cl. G01N 33/00

(52) U.S. Cl. CPC G01N 33/0022

(57) ABSTRACT

The invention describes a modular fluid sensing system comprising a fluid sensing terminal (150) and removable fluid channel units (101, 102, 103). The fluid channel units (101, 102, 103) comprise or can be combined with fluid treatment units as filters, sensors and seals. The fluid channel units (101, 102, 103) can be combined in accordance with the present needs of the user of the fluid sensing system. The invention therefore enables a reconfigurable channel system with reconfigurable measurement and treatment options within a fluid sensing system.

### FIELD OF THE INVENTION

The invention relates to fluid channel units for sensing a property of a fluid, a fluid sensing terminal which is adapted to receive such fluid channel units and a fluid sensing system comprising the fluid sensing terminal and the fluid channel units.

### BACKGROUND OF THE INVENTION

WO 2007/116130 discloses an apparatus for air purification in which the air to be purified is conducted during the purification process through more than one purification element, and in which the air to be purified is at least ionized and then conducted at least to a cold catalysis process taking place in a cold catalysis element equipped with a catalyst coating. In the cold catalysis process the catalyst coating is bombarded during UV radiation with a negatively charged electron shower and the air to be purified is oxidized at an essentially low temperature and the organic material in the air is turned into at least carbon dioxide and water vapor. The apparatus comprises one sensor at the inlet in order to monitor and measure the total pollution level of the air and a sensor at the outlet in order to monitor or and measure the purity of the air.

US 2010/0258211 A1 discloses a modular microfluidic system and a method of forming a microfluidic device by arranging the microfluidic assembly blocks on a base substrate. The purpose of the disclosure is to present a modular microfluidic system that allows for rapid prototyping.

US 2003/0012697 A1 discloses a microfluidic breadboard. The purpose of the disclosure is to provide an economical way of manufacturing a multipurpose lab-on-a-chip which can be used in the field of chemistry, biotechnology, chemical/environmental engineering. Swagelok: “Instrument Manifold Systems Instrument, Direct, and Remote-Mount Manifolds and Modular Systems”, 1 Feb. 2015 (2015 Feb. 1), pages 1-32, XP055287593 disclose a set of fluidic components which may be used to create a complex fluidic system.

WO 2015/112985 A1 discloses a microfluidic system comprising a microfluidic chip and a method of performing a chemical assay.

WO 2013/090188 A1 discloses a method for identifying and quantitatively analyzing an unknown organic compound in a gaseous medium which can be implemented using simple and inexpensive sensing equipment.

WO 2015/083079 A1 discloses a method and device for the analysis of a gas sample. A purpose of the disclosure is to present a device which can perform a fast and sensitive detection.

WO 2012/083432 A1 discloses a method for detecting an odor in a gas sample. The method comprises controlling the temperature of a gaseous composition to obtain a desired temperature and dividing the gaseous composition into a plurality samples having the same volume and the same constituents and measuring each of the samples with a different sensor adapted for measuring odors.

### SUMMARY OF THE INVENTION

It is an object of the present invention to provide a fluid sensing system able to detect multiple properties of a fluid in one device only.

According to a first embodiment a fluid channel unit for sensing a property of a fluid is provided. The fluid channel unit comprises at least a first fluid inlet, a first fluid outlet and at least a second fluid outlet. The fluid channel unit is arranged such that the fluid can flow from the first fluid inlet to the first fluid outlet and from the first fluid inlet to the second fluid outlet. The fluid channel unit is further arranged to be removably coupled to a fluid sensing terminal and at least a second fluid channel unit and a third fluid channel unit such that the fluid can flow from the first fluid outlet of the fluid channel unit to the first fluid inlet of the second fluid channel unit and such that the fluid can flow from the second fluid outlet of the fluid channel unit to the first fluid inlet of the third fluid channel unit. The fluid channel unit can thus be combined with other fluid channel units within the fluid sensing system in a changeable way.

The fluid channel units is arranged to enable a multitude of pathways within the fluid sensing terminal with a multitude of possible combinations of fluid treatment units and sensors which can be combined in order to determine multiple properties, contaminations or pollutants within a fluid. Such fluids may be investigated by means of a fluid sensing system comprising a fluid sensing terminal and several fluid channel units. Such a fluid sensing system can be configured such that multiple properties and especially pollutants in a fluid (air, gas, water et cetera.) can be detected. The fluid sensing system comprising the fluid channel units and the fluid sensing terminal may be used to investigate air quality, water quality or other fluids which may comprise admixtures of organic and inorganic substances (gases, liquids, particles and microorganisms). Fluid treatment units may be any device which can influence the property or properties of the fluid. An example of the fluid treatment unit may, for example, be a filter, a precipitator including an electrostatic precipitator, but also a UV emitting light source which is adapted to illuminate the fluid passing the fluid treatment unit. The sensor or sensors may be adapted to determine properties of the fluid as, for example, the composition of the fluid or a contamination of the fluid. Seals may be used to close one of the fluid inlet or outlets. The fluid may alternatively or in addition contain medication.

The fluid channel units may be arranged such that each of them contains preferably different microorganisms. Effectiveness of the medication can be investigated on different microorganisms by means of tailored configurations of fluid channel units. Fluid treatment units arranged between the different fluid channel units may be used to separate the different microorganisms.

The fluid channel unit may comprise a casing. The casing may be adapted to align and couple the fluid channel unit to the fluid sensing terminal. The casing may be further adapted to align and couple the fluid channel unit to the second and third fluid channel unit. The fluid channel units may be arranged as building blocks which can be coupled to each other and to the fluid sensing terminal. The fluid channel unit may comprise coupling structures which enable only a defined number of combinations to other fluid channel units. This may enable a simplified combination of different fluid channel units if each of the fluid channel units is characterized by specific fluid treatment and/or fluid sensing. The number of combinations of fluid channel units may be limited by means of the coupling structures. The coupling structures may comprise, for example, special combinations of pins and receptacles and the like such that the fluid channel unit can only be combined with predefined other fluid channel units and optionally only placed at the inlet or outlet of the fluid sensing terminal.

The fluid channel unit may comprise at least one socket arranged at the first fluid inlet, first fluid outlet or second fluid outlet. The socket is arranged to receive at least one device selected out of the group of fluid treatment unit, sensor or seal. The sockets may be arranged such that each socket can receive a fluid treatment unit, sensor or seal. Alternatively, the sockets may be arranged such that the socket can only receive only, for example, a fluid treatment unit or alternatively a sensor. A socket for receiving a fluid treatment unit may, for example, have a rectangular shape. As socket for receiving a sensor may, for example, have a triangular shape. It may in this case only possible to place a fluid treatment unit in the corresponding socket and a sensor in the other corresponding socket. Specified sockets in combination with corresponding treatment units, sensors and seals may have the advantage that only a limited number of combinations are possible in order to simplify the configuration of a fluid channel unit and finally a fluid sensing system. These specified sockets may be combined with specified coupling structures as described above in order to simplify the configuration of the fluid sensing systems.

The fluid channel unit may comprise at least one sensor. The sensor may be arranged in a channel connecting the first fluid inlet and the first fluid outlet, or wherein the sensor is arranged in a channel connecting the first fluid inlet and the second fluid outlet. The fluid channel unit may further comprise a sensor data interface being adapted to exchange a measurement result of the sensor with the fluid sensing terminal. The sensor data interface may be a wired interface extending to the outside of the fluid channel unit such that it can be contacted from the outside. The sensor data interface may alternatively be a wireless interface as, for example, an RFID interface integrated in the sensor. The sensor may be arranged in a way such that it can be activated or supplied with electrical power by means of the wired or wireless interface, for example, by means of an electromagnetic field provided by the fluid sensing terminal. The sensor may be arranged within one of the channels, at the inlet or at the outlets.

The fluid channel unit may comprise at least one fluid treatment unit. The fluid treatment unit may be arranged in a channel connecting the first fluid inlet and the first fluid outlet, or wherein the fluid treatment unit is arranged in a channel connecting the first fluid inlet and the second fluid outlet. The fluid treatment unit may be arranged within one of the channels, at the inlet or at the outlets. The fluid treatment unit may preferably be arranged after the branch to the first and second outlet. The fluid channel unit may further comprise one or more sensors which may be used to determine the properties of the fluid before passing the fluid treatment unit and/or after passing the fluid treatment unit.

The fluid channel unit may comprise at least one fluid pump for moving the fluid in or out of the fluid channel unit. The fluid pump may be arranged in a channel connecting the first fluid inlet and the first fluid outlet, or wherein the fluid pump is arranged in a channel connecting the first fluid inlet and the second fluid outlet. The fluid channel unit may comprise an electrical interface being adapted to supply electrical power to the fluid pump from the fluid sensing terminal. The fluid pump is preferably arranged such that the fluid is moved from the fluid inlet to the fluid outlets. Alternatively it may be arranged such that the fluid can be moved in both directions. The fluid pump may be any device which is adapted to move the fluid. Examples may be a fan for moving gases like air or liquid pumps.

The fluid channel unit further comprises at least one unit identification interface. The unit identification interface is adapted to exchange configuration information with the fluid sensing terminal regarding a fluid treatment unit, sensor or seal coupled to the fluid channel unit. A modular system may have the disadvantage that the user has to think about the configuration of the fluid sensing system. A detailed knowledge may be necessary in order to configure the fluid channel units comprising different combinations of fluid treatment units, sensors or seals such that the composition of the fluid can be determined or measured. The latter may be especially critical in cases in which, for example, pollutants or contamination can only be measured by differential measurements. The configuration information may enable to provide a construction manual to the user in order to combine the fluid channel units in accordance with the needs of the user. Furthermore, the configuration information may be used to check the combination of fluid channel units which may be placed in a coupling opening of the fluid sensing terminal. The unit identification interface may be a wired or wireless interface. The unit identification interface may alternatively be a kind of barcode which can be read by a corresponding reader unit integrated in a fluid sensing terminal. Such identification interfaces may also be arranged in individual treatment units, sensors or seals if they are configured to be removably coupled to the fluid channel unit. The identification interface may in combination with defined coupling be used to simplify the flexible use of the fluid channel units in order to determine the composition or properties of fluids. Standard configurations of combinations of fluid channel units may be performed in factory.

The fluid channel unit may comprise at least a second fluid inlet. The first fluid inlet, the second fluid inlet, the first fluid outlet and the second fluid outlet may be arranged such that first fluid inlet and the second fluid inlet can be used as first fluid outlet and second fluid outlet, and the first fluid outlet and the second fluid outlet can be used as first fluid inlet and second fluid inlet within the fluid sensing terminal. Such a symmetric approach of providing fluid channel units may have the advantage that the combination of the fluid channel units is simplified. Furthermore, electrical or data interfaces may be arranged such that the fluid channel units can be used in both direction meaning that the outlets can act that inlets and vice versa. The fluid channel units may be coupled or comprise fluid treatment units, sensors or seals as described above. Coupling structures, sockets or identification interfaces and the like may be used in the same way as described above. The fluid channel unit may comprise a third, fourth or more fluid inlets and/or a third, fourth or more fluid outlets respectively.

According to a further embodiment a fluid sensing terminal is provided. The fluid sensing terminal comprises a coupling opening for removably coupling at least three fluid channel units as described above. The coupling opening is shaped such that the at least three fluid channel units can be placed or inserted in the coupling opening of the fluid sensing terminal and such that appropriate fluid channel connections are made when the at least three fluid channel units are places in the coupling opening. The at least three fluid channel units can be removably coupled such that that the fluid can flow from at least one fluid inlet of the fluid sensing terminal via a first fluid inlet and a first fluid outlet of a first fluid channel unit to the first fluid inlet of the second fluid channel unit, and such that the fluid can flow from the fluid inlet of the fluid sensing terminal via the first fluid inlet and a second fluid outlet of the first fluid channel unit to the first fluid inlet of the third fluid channel unit. The fluid sensing terminal further comprises an evaluator. The evaluator is adapted to receive at least four sensor signals resulting from fluid property measurements of a fluid passing a first fluid outlet of the second fluid channel unit, a fluid passing a second fluid outlet of the second fluid channel unit, a fluid passing a first fluid outlet of the third fluid channel unit and a fluid passing a second fluid outlet of the third fluid channel unit. The fluid sensing terminal comprises at least one of a user interface for presenting at least a part of results of the fluid property measurements to a user of the fluid sensing terminal or a terminal data interface for exchanging data comprising at least a part of the results of the fluid property measurements.

The evaluator may further comprise a fluid pump controller which is arranged to control the fluid flow across the fluid channel units of the fluid sensing terminal. The user interface may comprise an acoustic or optical interface. The user interface may enable the user of the fluid sensing terminal to input data in order to define, for example, a configuration of the fluid sensing terminal. The terminal data interface may be arranged such that it can be coupled to another computing device in order to transfer the measurement data or a part thereof from the fluid sensing terminal to the computing device. Such a computing device may be any computer, laptop, mobile communication device and the like which can be used to present the measurement data to the user of the fluid sensing terminal. The terminal data interface may be further arranged to receive instructions and/or information. The terminal data interface may be any wired or wireless interface which can be used to transfer information from and to the fluid sensing terminal.

The evaluator may comprise one or more processing devices as processors or microprocessors and the like as well as one or more data storage devices as memory chips, optical memory device and the like.

The fluid sensing terminal may comprise at least one first fluid pump for moving the fluid via the at least one fluid inlet of the fluid sensing terminal to at least one fluid outlet of the fluid sensing terminal. Such integrated fluid pumps may be useful in case of passive fluid channel units which do not comprise active components as sensors, fluid pumps and the like.

The fluid sensing terminal further comprises at least one first sensor. The first sensor is adapted to measure the fluid property at least of the fluid passing the first fluid outlet of the second fluid channel unit, the fluid passing the second fluid outlet of the second fluid channel unit, the fluid passing the first fluid outlet of the third fluid channel unit and the fluid passing the second fluid outlet of the third fluid channel unit.

The first sensor may comprise different sensing areas in order to determine the properties of the fluids at the different fluid outlets. Alternatively, there may be a switching unit which is adapted to provide the fluid passing the different fluid outlets at different time sequences to the first sensor. The fluid sensing terminal may comprise one or more sensors at each of the fluid outlets of the fluid channel units in order to determine the composition or properties of the fluid. Such integrated sensors may be useful in case of passive fluid channel units which do not comprise active components as sensors, fluid pumps and the like.

The evaluator may be adapted to receive identification information from at least one device selected out of the group fluid channel unit, fluid treatment unit, sensor or seal. The fluid channel unit, fluid treatment unit, sensor or seal comprises in this case an identification interface which can provide information about the respective device. Such information enables the evaluator to check the configuration of the combined devices in order to determine which properties of the fluid can be detected to which extent.

According to a particular embodiment, at least four sensor signals provided to the evaluator are: 1) a first sensor signal coming from a first sensor which is located such that fluid property measurements of a fluid passing a first fluid outlet of the second fluid channel unit are provided to the evaluator; 2) a second sensor signal coming from a second sensor which is located such that fluid property measurements of a fluid passing a second fluid outlet of the second fluid channel unit are provided to the evaluator; 3) a third sensor signal coming from a third sensor which is located such that fluid property measurements of a fluid passing a first fluid outlet of the third fluid channel unit are provided to the evaluator; and 4) a fourth sensor signal coming from a fourth sensor which is located such that fluid property measurements of a fluid passing a second fluid outlet of the third fluid channel unit are provided to the evaluator. Thus, when the at least three channel units are coupled in the coupling opening and the first, second, third and fourth sensor are installed, all four sensors are coupled to the evaluator and provide their sensor signals.

The fluid sensing terminal may further comprise at least one terminal identification interface for each fluid channel unit. The terminal identification interfaces are adapted to receive the identification information via a corresponding unit identification interface of the fluid channel units.

The terminal identification interface may enable to determine which kind of fluid channel unit is coupled to a respective port of the fluid sensing terminal which comprises the terminal identification interface. The fluid sensing terminal may receive via the terminal identification interfaces configuration information of the fluid channel units. The terminal identification interface may be any kind of wired or wireless interface which enables detection of configuration information or exchange of configuration information of the fluid channel units.

The evaluator may be adapted to perform a configuration detection procedure for determining a configuration of the fluid sensing terminal coupled to the fluid channel units after coupling the fluid channel units with the fluid sensing terminal. Configuration information provided by the fluid channel units may be used in order to check the configuration or more precisely the arrangement of fluid channel units optionally comprising fluid treatment units, sensors or seals within the fluid sensing terminal. The configuration detection procedure may beneficially be supported by means of terminal identification interfaces which enable easy detection of the fluid channel unit coupled to the respective terminal identification interface. The fluid sensing terminal may alternatively or in addition comprise coupling structures in order to limit combinations between the fluid sensing terminal and the fluid channel devices. The coupling structures may be arranged as described above (e.g. male and female coupling structures). The evaluator may be further configured or adapted to receive configuration specifications describing the measurements which should be performed by means of the fluid sensing terminal. The evaluator may in this case check by means of the configuration information whether the configuration of the fluid channel units, fluid treatment units, sensors and seals is in accordance with the configuration specifications. The configuration specifications may be provided by means of a user of the fluid sensing terminal. The fluid sensing terminal may in this case comprise a user interface enabling the user to input the configuration specifications. The configuration specifications may alternatively be inputted by means of an application which may be stored on a, for example, mobile communication device and transfer the data by means of the terminal data interface as described above. The evaluator may be further adapted to receive via the terminal data interface information about the fluid. The evaluator may in this case propose a configuration of fluid channel units and optionally fluid treatment units, sensors and seals in order to determine defined properties or at least a part of the composition of the fluid. The configuration may be proposed to the user by means of the user interface or the mobile communication device. The user can accept the configuration and configure or reconfigure the fluid channel units such that the proposed properties of the fluid can be measured. The evaluator may in this case also be enabled to check by means of configuration information provided by the fluid channel units or other devices whether the configuration is in accordance with the proposed and accepted configuration.

The fluid sensing terminal may, for example, be an air quality detector. The air quality detector may comprise a wireless interface by means of which the air quality detector can receive air quality information from external measurement stations via the Internet. The evaluator of the air quality detector evaluates the received air quality information which may comprise information regarding certain pollutants. Such air quality information may comprise information regarding pollen. Pollen of different plants are characterized by a certain size distribution for which specific particle sizes may need to be detected. The evaluator may further check whether the current configuration of the air quality detector is arranged such that the pollutants can be determined. The air quality detector may inform the user by means of an optical user interface (e.g. display) about the air quality information and whether the air quality detector is arranged to determine or measure the pollutants. The evaluator of the air quality detector may be further arranged to present configuration or reconfiguration information by means of the display to the user if the air quality detector is not arranged to determine or measure the pollutants specified in the air quality information. The configuration or reconfiguration information gives advice to the user how to configure or reconfigure the air quality detector such that the air quality detector is arranged to measure the pollutants. The air quality detector may preferably be arranged to check the configuration of the air quality detector by means of information provided by the fluid (air) channel units added to the air quality detector. The air channel units comprise in this case preferably a fixed configuration of fluid treatment units (e.g. filters), sensors and seals. Such a fixed configuration enables a simple check of the configuration of the air quality detector especially by means of a corresponding terminal identification interface.

The method of checking the configuration of the air quality detector and the proposal to configure or reconfigure the air quality detector may alternatively be performed by means of the computing device such as a mobile communication device. The mobile communication device may in this case receive configuration data from the air quality detector and air quality information via the Internet. The mobile communication device may in this case comprise an application (software program) which performs the check of the configuration of the air quality detector in view of the air quality information as described above.

The example given by means of the air quality detector may be extended to each kind of fluid sensing terminal or fluid sensing system.

According to a further embodiment a fluid sensing system is provided. The fluid sensing system comprises at least one fluid sensing terminal as described above and at least three fluid channel units as described above. The fluid sensing system can be configured such that multiple properties and especially pollutants in a fluid (air, gas, water et cetera.) can be detected. The fluid sensing system comprising the fluid channel units and the fluid sensing terminal may be used to investigate air quality, water quality or other fluids which may comprise admixtures of organic and inorganic substances (gases, liquids, particles and microorganisms).

According to a further embodiment an air purifier is provided. The air purifier comprises at least one fluid sensing terminal which is adapted to receive fluid channel units as described above.

According to a further embodiment a computer program product is presented. The computer program product comprises code means which can be saved on at least one memory device comprised by the fluid sensing terminal or communication device as described above (e.g. an EEPROM, hard disk or solid state disk), wherein the code means being arranged such that the method of configuring or reconfiguring the fluid sensing system as described above can be executed by means of at least one processing device comprised by the fluid sensing terminal or communication device as described above. The processing device may comprise one or more processors or microprocessors or controllers. The method may also be performed by means of a system comprising the fluid sensing terminal and the communication device such that parts of the method steps are performed either by means of the fluid sensing terminal or the communication device especially mobile communication device.

It shall be understood that a preferred embodiment of the invention can also be any combination of the dependent claims with the respective independent claim. Further advantageous embodiments are defined below.

### BRIEF DESCRIPTION OF THE DRAWINGS

These and other embodiments of the invention will be apparent from and elucidated with reference to the embodiments described hereinafter.

The invention will now be described, by way of example, based on embodiments with reference to the accompanying drawings.

In the drawings:

FIG. 1 shows a principal sketch of a cross-section of a first embodiment of a fluid channel unit;

FIG. 2 shows a principal sketch of a top view of an arrangement of three fluid channel units;

FIG. 3 shows a principal sketch of a cross-section of a first embodiment of a fluid sensing system;

FIG. 4 shows a principal sketch of a top view of an arrangement of six fluid channel units;

FIG. 5 shows a principal sketch of a cross-section of a second embodiment of a fluid channel unit;

FIG. 6 shows a principal sketch of a cross-section of a second embodiment of a fluid sensing system;

FIG. 7 shows a principal sketch of a cross-section of a third embodiment of a fluid sensing system;

FIG. 8 shows a principal sketch of a cross-section of a fourth embodiment of a fluid sensing system;

FIG. 9 shows a principal sketch of a cross-section of a first embodiment of a sensor module;

FIG. 10 shows a principal sketch of a method of fluid property detection;

FIGS. 11 and 12 show a principal sketch of a cross-section of a fifth embodiment of a fluid sensing system; and

FIG. 13 shows a principal sketch of a first embodiment of an air purifier.

### DETAILED DESCRIPTION OF EMBODIMENTS

Various embodiments of the invention will now be described by means of the Figures.

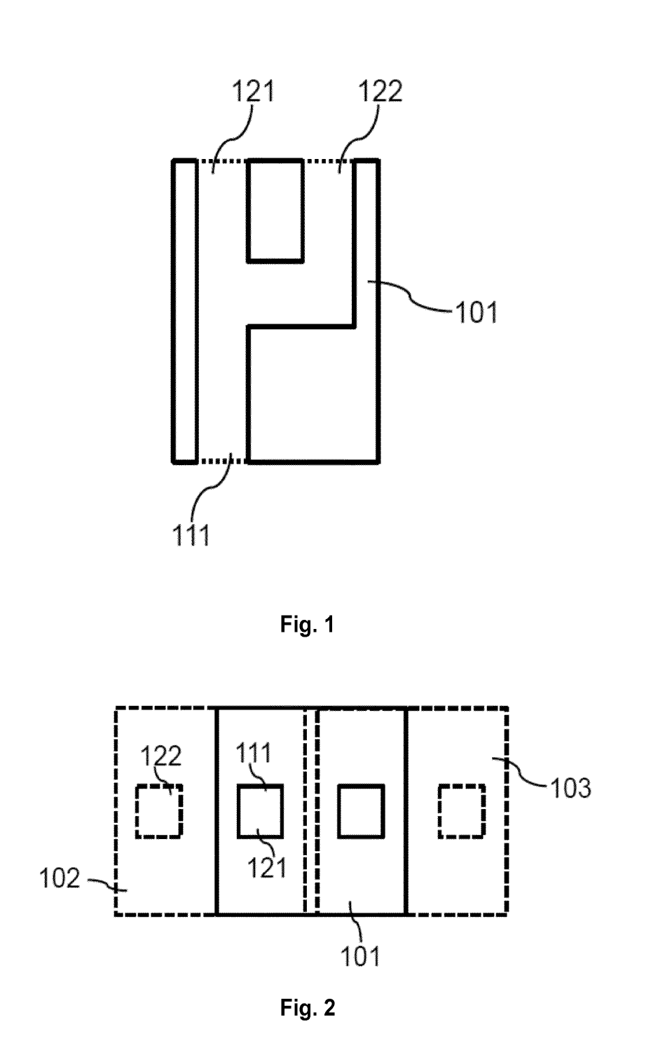

FIG. 1 shows a principal sketch of a cross-section of a first embodiment of a fluid channel unit. FIG. 1 shows a first fluid channel unit 101 with a first fluid inlet 111, a first fluid outlet 121 and a second fluid outlet 122. The first fluid inlet 111 is connected by means of a channel with the first fluid outlet 121 and the second fluid outlet 122. The channel comprises a Y-shaped branch between the first fluid inlet 111 and the first and the second fluid outlet 121, 122.

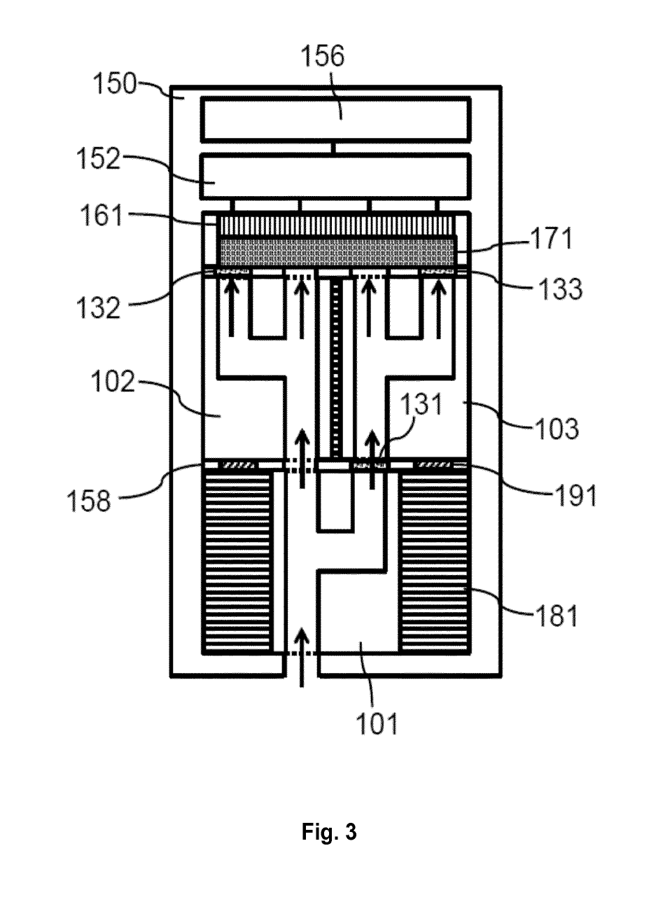

FIG. 2 shows a principal sketch of a top view of an arrangement of three fluid channel units. The first fluid channel unit 101 depicted by means of solid lines is arranged below a second fluid channel unit 102 and a third fluid channel unit 103 which are depicted by dashed lines. The first fluid inlet 111 of the first fluid channel unit 101 is arranged directly below the first fluid outlet 121 as shown in FIG. 1. The first fluid outlet 121 of the first fluid channel unit 101 is coupled to a first fluid inlet 111 of the second fluid channel unit 102 and the second fluid outlet 122 of the first fluid channel unit 101 is coupled to the first fluid inlet 111 of the third fluid channel unit 103. Fluid entering the first fluid channel unit 101 via the first fluid inlet 111 of the first fluid channel unit can therefore be spread by means of the first and the second fluid outlet 121, 122 of the first fluid channel unit 101 to four fluid outlets, the first and the second fluid outlet 121, 122 of the second fluid channel unit 102 and the first and the second fluid outlet 121, 122 of the third fluid channel unit 103. A fluid can be distributed by means of the fluid channel units in a flexible and simple way.

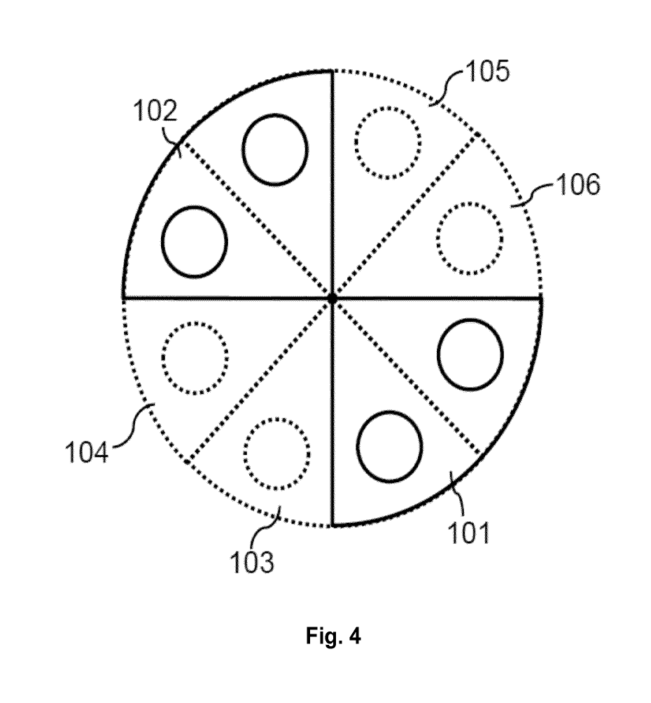

FIG. 3 shows a principal sketch of a cross-section of a first embodiment of a fluid sensing system. The fluid sensing system comprises a fluid sensing terminal 150 and three fluid channel units 101, 102, 103 which are removably coupled to the fluid sensing terminal 150 in a coupling opening. The arrangement of the fluid channel units 101, 102, 103 within the fluid sensing terminal 150 is similar as the arrangement discussed with respect to FIG. 2. The coupling opening of the fluid sensing terminal 150 comprises a separator with four holes in which seals, sensors or fluid treatment units can be placed. The first fluid channel unit 101 is placed within the coupling opening by means of two dummy units 181 such that the fluid can enter the first fluid channel unit 101 via the first fluid inlet 111 and leave the first fluid channel unit 101 via the first fluid outlet 121 which is coupled to an opening of the separator 158 and the other second fluid outlet 122 which is coupled to a first fluid treatment unit 131 placed in the corresponding hole of separator 158. The first fluid treatment unit 131 is in this case a filter for filtering the fluid with respect to one defined substance. The unfiltered fluid enters the second fluid channel unit 102 via the first fluid inlet 111. The unfiltered fluid leaves the second fluid channel unit 102 via the first fluid outlet 121 and via the second fluid outlet 122 which is coupled to a second fluid treatment unit 132. The filtered fluid enters the third fluid channel unit 102 via the first fluid inlet 111. The filtered fluid leaves the third fluid channel unit 103 via the first fluid outlet 121 and via the second fluid outlet 122 which is coupled to a third fluid treatment unit 133. The second and the third fluid treatment unit 132, 133 are placed within holes of a separator similar as separator 158. The configuration of the fluid sensing system can in this case be configured or reconfigured by using different fluid treatment units and placing the fluid treatment units at different positions within the coupling opening comprising separator 158. The fluid leaving the different fluid outlets of the second and the third fluid channel unit 102, 103 are received by the first sensor 171 which comprises a sensor array for detecting the composition of the fluid leaving the different fluid outlets. The fluid sensing terminal 150 further comprises a first fluid pump 161 which pumps the fluid from the first fluid inlet 111 of the first fluid channel unit 101 to a common fluid outlet behind the first sensor 171. The fluid pump 161 is controlled by means of evaluator 152 which is comprised by the fluid sensing terminal 150. The evaluator 152 further receives measurement data from the first sensor 171 and determines the composition or more generally one or more property of the fluid by means of the measurement data. The property or properties of the fluid is presented to the user by means of user interface 156 which is comprised by the fluid sensing terminal.

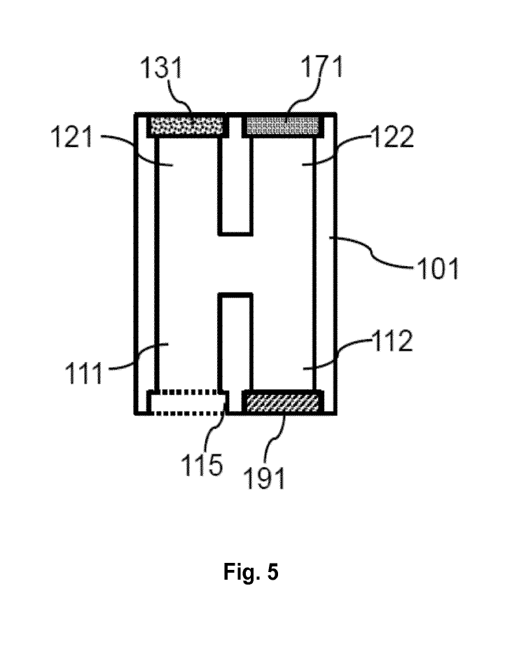

FIG. 4 shows a principal sketch of a top view of an arrangement of six fluid channel units. FIGS. 2 and 3 show two-dimensional arrangements of fluid channel units. FIG. 4 shows an extension of this principle to a three dimensional arrangement. The fluid channel units or more precise the casing are in this case arranged as a quarter of a cylinder like pieces of a cake. Each fluid channel unit comprises one fluid inlet and two fluid outlets at the parallel surfaces of the quarters. This arrangement enables a coupling of a first fluid channel unit 101 and the second fluid channel unit 102 to 4 fluid channel units 103, 104, 105 and 106. The first fluid channel unit 101 is coupled to the third fluid channel unit 103 and the sixth fluid channel unit 106, wherein the second fluid channel unit 102 is coupled to the fourth fluid channel unit 104 and the fifth fluid channel unit 105. The third, fourth, fifth and sixth fluid channel unit 103, 104, 105 and 106 are arranged in a cylinder shape.

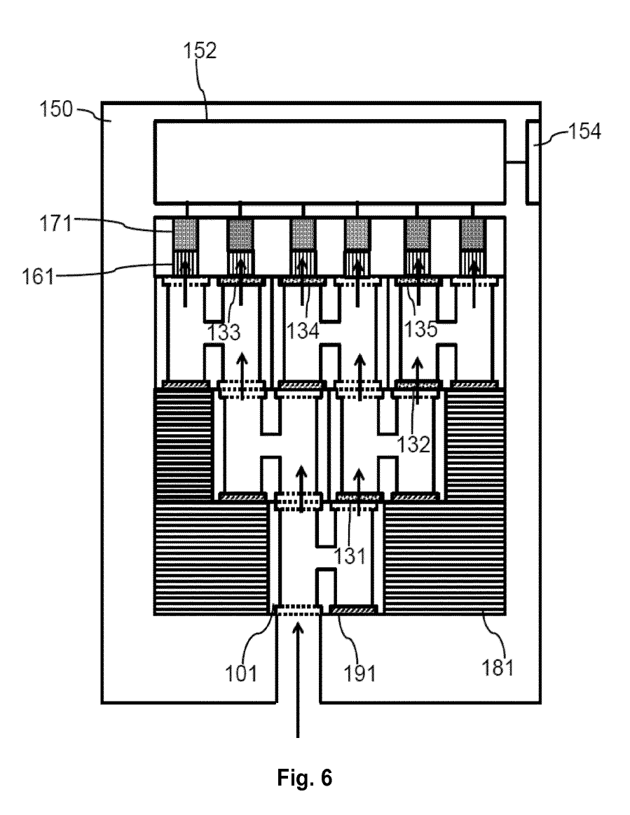

FIG. 5 shows a principal sketch of a cross-section of a second embodiment of a fluid channel unit. The second embodiment of the first fluid channel unit 101 comprises an H-shaped channel configuration. The first fluid channel unit 101 or more precise the casing of the first fluid channel unit 101 is in this case preferably a rectangular block wherein a first and a second fluid inlet 111, 112 of the H-shaped channel configuration are arranged on one surface of the rectangular block and a first and a second fluid outlet 121, 122 are arranged on an opposite surface of the rectangular block. Each of the fluid inlets is connected to each of the outlets by means of the H-shaped channel configuration. The symmetry of the configuration enables to use the inlets as outlets and vice versa. The second embodiment of the first fluid channel unit 101 further comprises four sockets 115 at each of the fluid inlet and outlets which are arranged to receive a cylinder-shaped first fluid treatment unit 131, first sensor 171 or first seal 191. Each of the fluid channel units can therefore be individually configured by means of fluid treatment units, sensors or seals which can be placed in the sockets.

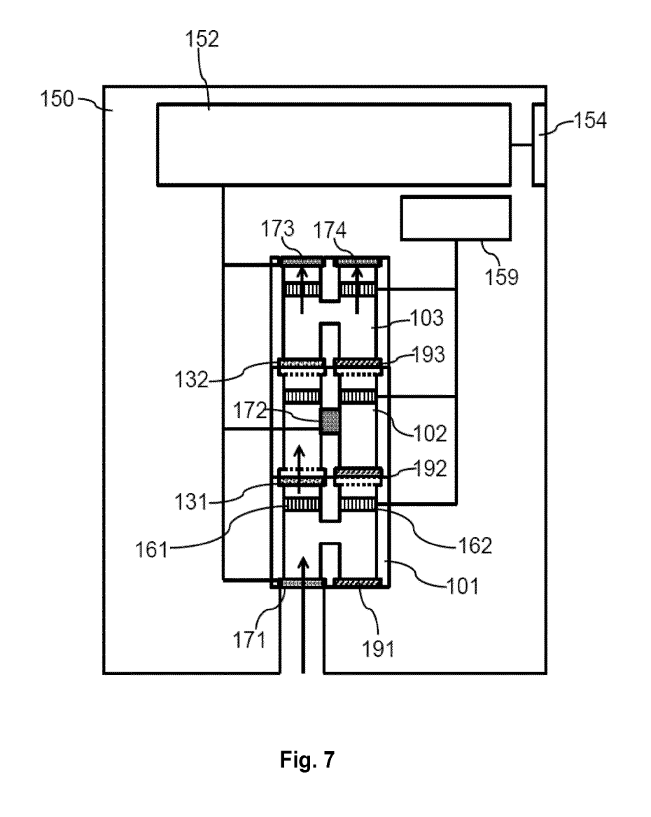

FIG. 6 shows a principal sketch of a cross-section of a second embodiment of a fluid sensing system. The overall arrangement of the fluid sensing system according to the second embodiment is similar to the arrangement of the first embodiment of the fluid sensing system as discussed with respect to FIG. 3. The second embodiment is more flexible because it comprises three rows of fluid channel units wherein the first embodiment comprises only two rows of fluid channel units. Furthermore, the second embodiment of the fluid sensing system comprises fluid channel units according to the second embodiment as described with respect to FIG. 5. The fluid sensing system is configured in this case as air purity detector. The air purity detector comprises a fluid sensing terminal 150 which is arranged in this case as the air purity terminal which can receive six fluid channel units which are arranged as air channel units. The six air channel units are stacked upon each other such that air entering the air purity detector via one outlet is split in six different air path by means of the fluid channel units. The air flows leaving the air channel units via the six air outlets of the upper three air channel units are treated by means of a first fluid treatment unit 131, a second fluid treatment unit 132, a third fluid treatment unit 133, a fourth fluid treatment unit 134 and a fifth fluid treatment unit 135 such that each airflow of the fixed outflows may have different properties or a different composition depending on the initial composition or properties of the air at the air inlet. The air may, for example, be contaminated by means of five different pollutants. Filter 131 is in this case arranged to filter pollutants A and B. Filter 132 is arranged to filter pollutants C and D. Filter 133 is arranged to filter pollutant A. Filter 134 is arranged to filter pollutant C. Filter 135 is arranged to filter pollutant E. The air channel units and the filters are in this case arranged such that a first air flow comprises all pollutants, a second air flow comprises four pollutants, a third air flow comprises three pollutants, a fourth air flow comprises two pollutants, a fifth airflow comprises one pollutant and a sixed airflow comprises no pollutant. Each of the outlets of the upper row of air channel units are coupled to corresponding fans (e.g. first fluid pump 161 coupled to the airflow with all pollutants). Each airflow is analyzed by a corresponding sensor (e.g. first sensor 171 which is coupled to the airflow with all pollutants). The sensors are arranged to determine all pollutants. A qualitative measurement can therefore be made by means of a differential analysis of the measurement results provided by means of the six sensors which is performed by means of evaluator 152. The result of the analysis is distributed by means of terminal data interface 154. The terminal data interface 154 is in this case a wireless Bluetooth interface such that the results can be received by means of a mobile communication device with a corresponding software application. The use of the mobile communication device can visualize the results of the analysis by means of the display of the mobile communication device.

Each fluid treatment unit 131, 132, 133, 134, 135 (filters) may comprise, for example, an RFID identifier or a data interface. The evaluator 152 may in this case be arranged to identify the respective fluid treatment unit 131, 132, 133, 134, 135. The sensors may comprise such identifier, too. Such a configuration may enable the evaluator 152 to determine the configuration of the fluid sensing system. The evaluator 152 may be further arranged, either by means of identifiers and data interfaces to determine which of the inlets and/or outlets of the fluid channel units are sealed. The fluid sensing terminal 150 may be further adapted to determine the number and kind of fluid treatment units within a flow path of the sensor, for example, by means of corresponding flow or pressure sensors. There may be a test run available to test the configuration of the fluid sensing system comprising the fluid sensing terminal 150, the fluid channel units, the fluid treatment units et cetera.

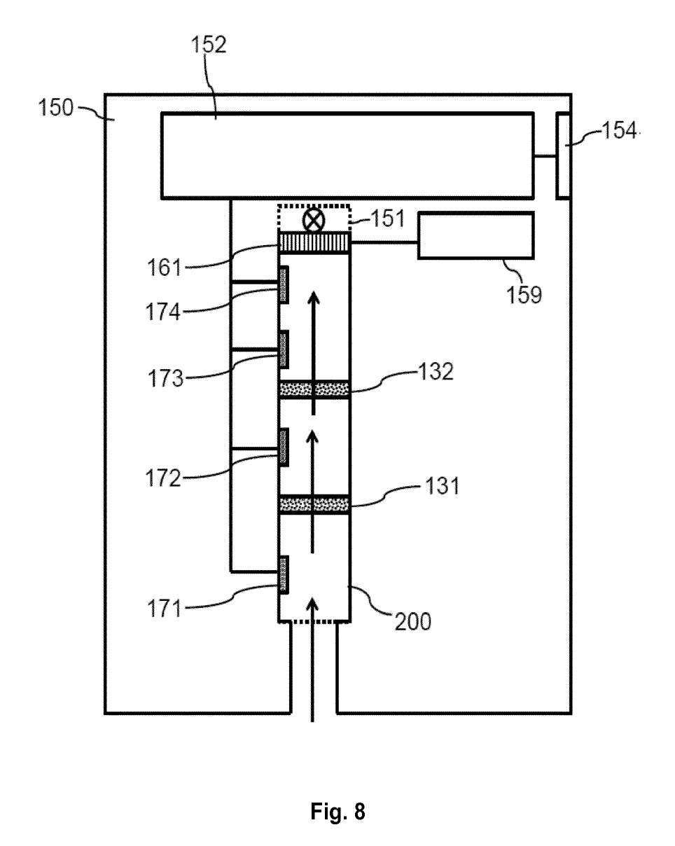

FIG. 7 shows a principal sketch of a cross-section of a third embodiment of a fluid sensing system. The fluid sensing system comprises a fluid sensing terminal 150 and three fluid channel units 101, 102, 103 wherein the fluid channel units are arranged in a similar way as the fluid channel units described in FIG. 5. The fluid sensing terminal 150 comprises a fluid pump controller 159 which is arranged to control the fluid pumps 161, 162 comprised by the fluid channel units 101, 102, 103. The fluid sensing terminal 150 further comprises an evaluator 152 which is connected by means of a wired interface to a first sensor 171, a second sensor 172, a third sensor 173 and a fourth sensor 174 which are coupled to the fluid channel units 101, 102, 103. The evaluator 152 is coupled to a terminal data interface 154 which is arranged to transfer data provided by evaluator 152 by means of the wired interface. A first fluid channel unit 101 comprises an H-shaped channel structure wherein the two channels after the H shape branch near to the fluid outlets comprise the first fluid pump 161 and a second fluid pump 162. A first fluid inlet of the first fluid channel unit 101 comprises a socket in which the first sensor 171 is placed. A second fluid inlet of the first fluid channel unit 101 comprises a socket in which a first seal 191 is placed. The first fluid outlet comprises a socket in which a first fluid treatment unit 131 is placed. The first and the second fluid outlet of the first fluid channel unit are coupled to a first and a second fluid inlet of a second fluid channel unit 102. The first fluid inlet of the second fluid channel unit 102 is open (no seal, fluid treatment unit, or sensor) and coupled to the first fluid outlet of the first fluid channel unit 101. The second fluid inlet of the second fluid channel unit 102 comprises a socket in which a second seal 192 is placed such that no fluid can flow via the second fluid outlet of the first fluid channel unit 101 and the second fluid inlet of the second fluid channel unit 102. The second fluid channel unit 102 further comprises a second sensor 172 which is arranged in the horizontal connection of the H-shaped channel structure. The second sensor 172 is arranged such that fluid can pass the horizontal connection. The two channels of the second fluid channel unit 102 comprise after the H shape branch near to the fluid outlets two fluid pumps as discussed with respect to the first fluid channel unit 101. The first and the second fluid outlet of the second fluid channel unit 102 are open. The first fluid outlet of the second fluid channel unit 102 is coupled to a first fluid inlet of a third fluid channel unit 103. The first fluid inlet of the third fluid channel unit 103 comprises socket in which a second fluid treatment unit 132 is placed. The second fluid outlet of the second fluid channel unit 102 is coupled to the second fluid inlet of the third fluid channel unit 103. The second fluid inlet of the third fluid channel unit 103 comprises a socket in which a third seal 193 is placed such that no fluid can flow via the second fluid outlet of the second fluid channel unit 102 and the second fluid inlet of the third fluid channel unit 103. The fluid passing the second fluid treatment unit 132 is further pumped by means of two fluid pumps which are arranged after the H shape branch near to the fluid outlet of the surf fluid channel unit 103 as discussed with respect to the first fluid channel unit 101. The fluid leaves the third fluid channel unit 103 via the first and the second fluid outlet. The first fluid outlet of the third fluid channel unit 103 comprises a socket in which a third sensor 173 is placed. The second fluid outlet of the third fluid channel unit 103 comprises a socket in which a fourth sensor 174 is placed. The evaluator 152 controls measurement intervals of the sensors 171, 172, 173, 174. The fluid sensing system as described in FIG. 7 may in a special embodiment be arranged to determine contamination of air by means of volatile organic compounds (VOC). The first, second, third and fourth sensor 171, 172, 173, 174 are arranged as semiconductor-based metal oxide (MOX) sensors which are very sensitive to a plurality of organic compounds, for this reason they are applied for sensing VOC in air. The first sensor 171 detects all VOCs that can be detected using the MOX principle (TVOC). After absorption of (Form)aldehyde by means of the first fluid treatment unit 131 which is arranged as an (Form)aldehyde filter, the second sensor 172 detects the TVOC concentration minus the (Form)aldehyde concentration which has been removed. The difference between the two signals is equal to the signal due to the detection of (Form)aldehyde. In exactly the same manner, the third sensor detects the TVOC signal minus the (Form)aldehyde signal minus the signal due to all the VOCs that are removed by second fluid treatment unit 132 which is arranged as activated carbon filter. The third sensor 173 can, in principle, also be used for recalibration, as it will hardly be exposed (if at all) to any remaining gases like, for example, silanes. By operating the fourth sensor 174 in a dedicated recalibration mode only (i.e. only heating it during this mode), it can be checked whether sensors 1-3 have been exposed to other gases that have influenced the sensitivity of these sensors, due to precipitation of e.g. SiOx on top of the sensitive layers which may be caused by decomposition of Silanes at the working temperatures of the MOX sensors in the temperature range of 300-400° C. This shows up in a significant change in relative signal between sensors 3 and 4, also in the absence of any VOCs. This can be used to determine the End of Life (EoL) of the sensor and therefore of the respective fluid channel unit 101, 102, 103.

The signals obtained this way need to be translated in information that is of relevance for the consumer. The latter is done by means of evaluator 152. The total VOC signal is the weighed sum (by the sensor sensitivity and the pollutant concentration) of each organic pollutant contributing to the sensor signal. Transfer functions convert this into a number that is indicative of the total VOC concentrations, weighed for example by their health impact. Further downstream, information concerning subsets of pollutants is obtained, to be converted into numbers in the same way as depicted in table 1:

TABLE 1

| Sensor 1 | Sensor 2 | Sensor 3 | Sensor 4 |
|---|---|---|---|
| Largest | Intermediate | Signal on sensor 3 | Signal on sensor 4 |
| signal | signal | likely to be | likely to |
| | | (close to) zero | be (close to) |
| | | | zero |
| TVOCs | Sensor 1- | Sensor 1-sensor 3: | Sensor 4-sensor 3: |
| | sensor 2: | TVOCs (in combination | EoL indication |
| | Aldehydes | with sensor 1 signal | |
| | | only: indication of | |
| | | differential aging) | |
| | | Sensor 2-sensor 3: all | |
| | | TVOCs not being | |
| | | (Form)aldehydes (lik BTX) | |

The table also shows that the differential signal between sensor 3 and 1 and the signal from sensor 1 in principle should be identical. In first order, this can be used to recalibrate sensor 1. Additional information on aging is obtained by comparing sensor 3 and sensor 4. Any difference in aging behavior is due to the fact that sensor 4 is used much less frequently than sensor 3. When this difference is significant, then the system or the corresponding fluid channel unit has reached its End of Life.

When either the formaldehyde or VOC subgroup (BTX-benzene, toluene, and the three xylene isomers) detection is not needed, to save cost the corresponding filter and MOX sensor can be omitted while retaining the functioning of the sensor system. This modularity could be designed such that the user can add the missing functionality post-production by using different fluid channel units. This modularity can further be easily enabled by means of removing the corresponding fluid treatment unit or units and/or sensor or sensors within the modular arrangement as shown in FIG. 7.

The sensors ideally are operated by means of evaluator 152 or independent control unit intermittently, to increase their operational life time (most of the time they are off). In addition, the air flow through the sensing system can be interrupted by means of fluid pump controller 159. The measurement frequency can be increased when the VOC concentrations are changing rapidly. This can be checked in the regular sensing scheme, also intermediate measurements of, for example, the TVOC concentrations (i.e. only using the first sensor 171) can be used to obtain this information.

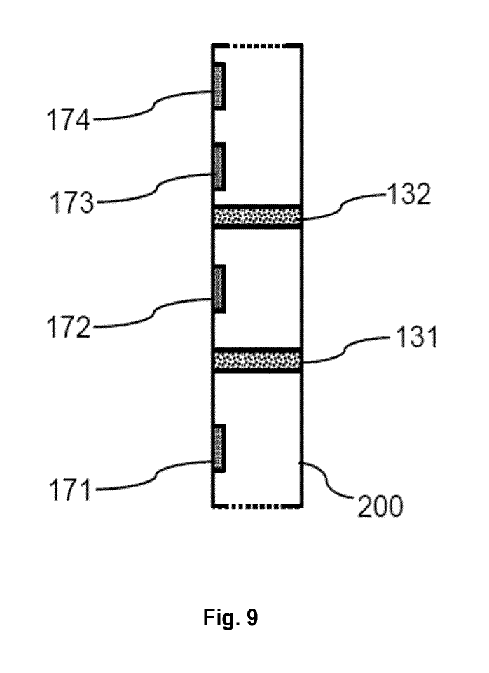

FIG. 8 shows a principal sketch of a cross-section of a fourth embodiment of a fluid sensing system. The arrangement of the fluid sensing system in FIG. 8 is similar to the arrangement of the fluid sensing system shown in FIG. 7. The three fluid channel units 101, 102, 103 shown in FIG. 7 are replaced by one sensor module 200. The sensor module 200 comprises a fluid (air) inlet with the first MOX sensor 171 in a first chamber. The first chamber is separated by means of the first treatment unit 131 which is again arranged as a (Form)aldehyde filter from the second chamber comprising the second MOX sensor 172 which detects the TVOC concentration minus the (Form)aldehyde concentration. The second chamber is separated by means of the second fluid treatment unit 132 which is again arranged as activated carbon filter. The third MOX sensor 173 can also be used for recalibration as discussed above with respect to FIG. 7. The sensor module 200 can be replaced at the end of life which can be determined by means of the fourth MOX sensor 174 which is only used to check whether the first, the second, and the third MOX sensors 171, 172, 173 still work properly as discussed above. The fluid is moved by means of the first fluid pump 161 which is arranged as a fan at the outlet of sensor module 200. The fan is controlled by means of fluid pump controller 159 which is comprised by the fluid sensing terminal 150. The fluid sensing terminal 150 further comprises electrical contacts to drive the sensors (e.g. heating up to temperatures between 300 and 400° C.) and to read out measurement results which are analyzed by means of evaluator 152. The results of the analysis of the measurement results can be read out by means of terminal data interface 154 which comprises a wired and wireless interface this case. The fluid (air) leaves the fluid sensing terminal 150 via fluid outlet of the terminal 151.

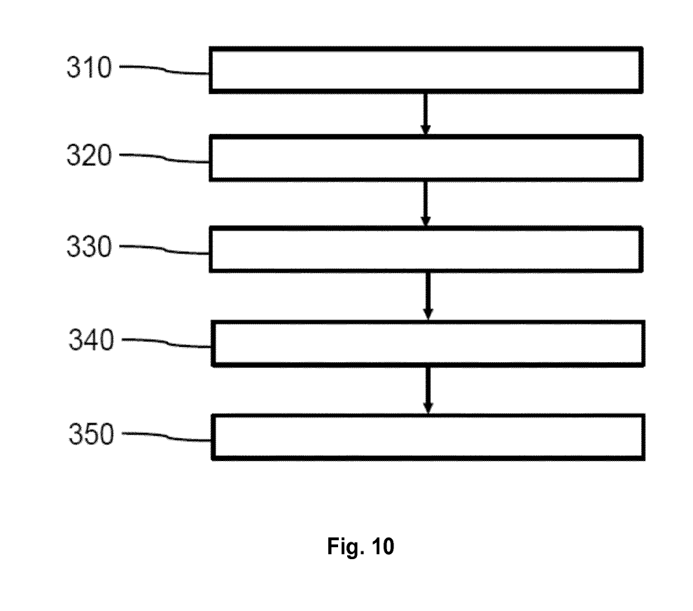

FIG. 9 shows a principal sketch of a cross-section of a first embodiment of a sensor module 200. The configuration is the same as discussed with respect to FIG. 8. The sensor module 200 is arranged as a disposable which can be replaced at the end of life.

FIG. 8-9 show a sensor module 200 for measuring contamination of air with volatile organic compounds. The sensor module 200 comprises a first metal oxide sensor 171, a second metal oxide sensor 172, a third metal oxide sensor 173 and the fourth metal oxide sensor 174. The first metal oxide sensor is arranged to detect all volatile oxide compounds that can be detected using metal oxide sensor (TVOC). The first metal oxide sensor 171 is separated by means a first fluid treatment unit 131 from the second metal oxide sensor 172. The first fluid treatment unit 131 is arranged as an (Form)aldehyde filter. The second metal oxide sensor 172 is arranged to detect the TVOC concentration minus the (Form)aldehyde concentration. The second metal oxide sensor 171 is separated by means of a second fluid treatment unit 132 from the third metal oxide sensor 173 and the fourth metal oxide sensor 174. The second fluid treatment unit 132 is arranged as an activated carbon filter such that essentially all volatile organic compounds are removed by means of the second fluid treatment unit 132. The third metal oxide sensor 173 is arranged to detect the TVOC signal minus the (Form)aldehyde signal minus the signal due to all the volatile oxide compounds that are removed by the activated carbon filter. The fourth sensor 174 is arranged to be operated in a pulsed mode in order to determine the End of Life (EoL) of the sensor module 200 as described above. The sensor module 200 can be modified by removing one of the sensors and or one of the fluid treatment units as discussed above with respect to FIG. 7. The fluid sensing terminal 150 as shown in FIG. 8 comprises a coupling opening for removably coupling the sensor module 200. The fluid sensing terminal 150 further comprises a first fluid pump 161 for pumping or moving the air through the sensor module 200. The fluid sensing terminal 150 further comprises an evaluator 152 which is optionally arranged to drive the sensors 171, 172, 173, 174 (may alternatively be autonomous devices) and to read out measurement data provided by the sensors. The fluid sensing terminal 150 further comprises at least one of a user interface 156 for presenting at least a part of results of the fluid property or contamination measurements to a user of the fluid sensing terminal 150 or a terminal data interface 154 for exchanging data comprising at least a part of the results of the fluid contamination or property measurements.

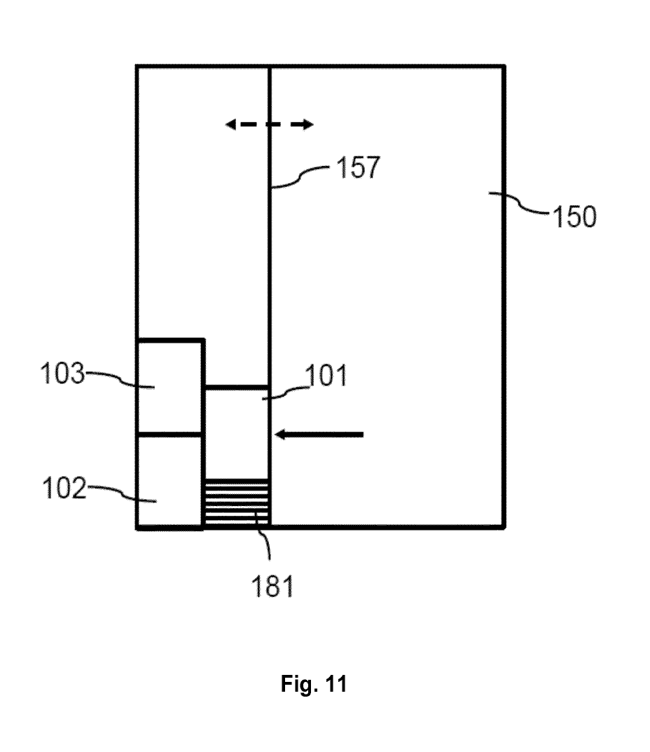

FIG. 10 shows a principal sketch of a method of fluid especially air property detection. In step 310 is a total concentration of volatile oxide compounds (TVOC) detected that can be detected using a first metal oxide sensor 171. In step 320 are (Form)aldehydes filtered. In step 330 is the TVOC concentration minus the (Form)aldehyde concentration detected by means of a second metal oxide sensor 172. In step 340 essentially all remaining volatile oxide compounds are removed by means of an activated carbon filter. In step 350 is the TVOC signal minus the (Form)aldehyde signal minus the signal due to all the volatile oxide compounds that are removed by the activated carbon filter detected by means of a third metal oxide sensor 173. The third metal oxide 173 is used in an optional further step to recalibrate the first and the second metal oxide sensor 171, 172 as discussed above. In a further optional step is the performance of the first, the second and the third metal oxide sensor 171, 172, 173 checked by means of a fourth metal oxide sensor 174 which measures the TVOC signal minus the (Form)aldehyde signal minus the signal due to all the volatile oxide compounds that are removed by the activated carbon filter in a pulsed mode such that the fourth metal oxide sensor 174 is used less frequent as the first, the second and the third metal oxide sensor 171, 172, 173 (see table 1 and the corresponding description).

A computer program product comprises code means which can be saved on at least one memory device comprised by the fluid sensing terminal or communication device as described above (e.g. an EEPROM, hard disk or solid state disk), wherein the code means being arranged such that the method as described in FIG. 10 and the corresponding description can be executed by means of at least one processing device comprised by the fluid sensing terminal or communication device as described above. The processing device may comprise one or more processors or microprocessors or controller. The method may also be performed by means of a system comprising the fluid sensing terminal and the communication device such that parts of the method steps are performed either by means of the fluid sensing terminal or the communication device especially mobile communication device.

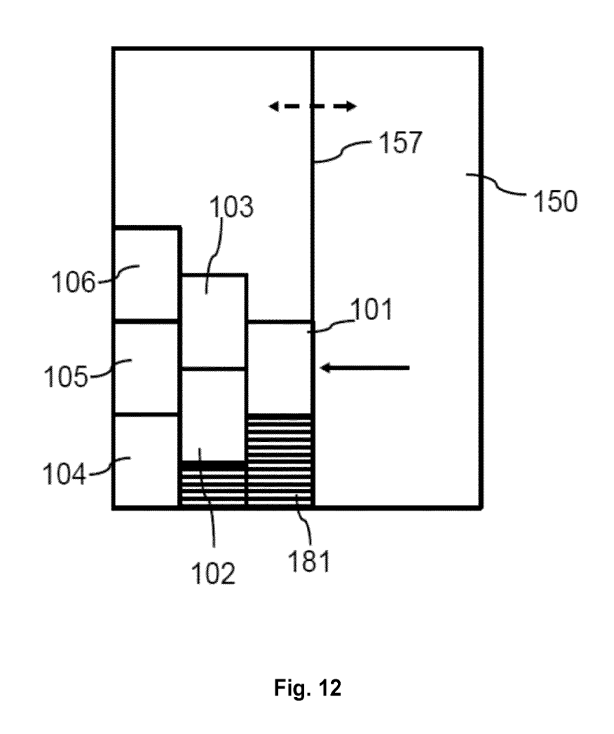

FIGS. 11 and 12 show a principal sketch of a cross-section of a fifth embodiment of a fluid sensing system comprising the fluid sensing terminal 150. The fluid sensing system is a scalable system which can be adapted by means of a movable wall 157 indicated by means of the double arrow. FIG. 11 shows a configuration in which three fluid channel units 101, 102, 103 as described, for example, in FIGS. 1 and 5 and the corresponding description are arranged in a similar way as shown in FIG. 3. The movable wall can be moved as shown in FIG. 12 such that three additional fluid channel units 104, 105, 106 can be added. The fluid channel units 101, 102, 103, 104, 105, 106 are in this case arranged similar as shown and discussed with respect to FIG. 6. Further fluid channel units can be added by moving movable wall. The fluid sensing system can therefore be adapted by choosing fluid channel units, sensors, fluid treatment unit and seal and especially the number of fluid channel units. The air inlet or more general fluid inlet which is indicated by the solid arrow can be adapted by means of holes in the movable wall 157 which can be closed by means of seals or by removing a seal. The fluid channel units can be arranged by means of dummy units 181.

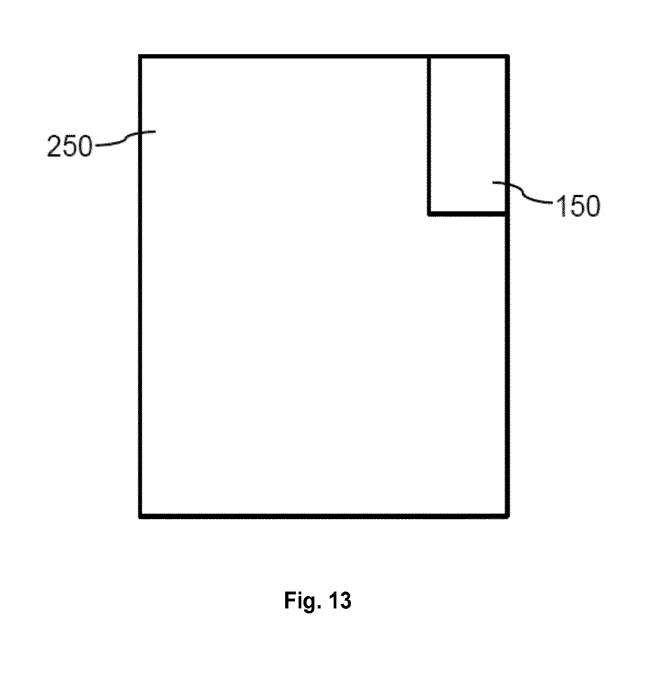

FIG. 13 shows a principal sketch of a first embodiment of an air purifier 250. The air purifier comprises a fluid sensing system with fluid sensing terminal 150 according to one of the embodiments discussed above. The fluid sensing system is preferably a scalable fluid sensing system as described in FIGS. 11 and 12 and the corresponding description. It may therefore be possible to adapt the fluid sensing system in accordance with up-to-date air pollution information which may be monitored by means of professional sensor stations. The fluid sensing system may further be arranged such that the air flowing in the air purifier can be checked and the air leaving the air purifier can be checked.

It is a basic idea of the present invention to provide a modular fluid sensing system comprising a fluid sensing terminal 150 and removable fluid channel units 101, 102, 103. The fluid channel units 101, 102, 103 comprise or can be combined with fluid treatment units as filters, sensors, seals and the like. The fluid channel units 101, 102, 103 can be combined in accordance with the present needs of the user of the fluid sensing system. The invention therefore enables a reconfigurable channel system with reconfigurable measurement and treatment options within a fluid sensing system. The fluids may be gases like air, liquids like water and the like. The modular fluid sensing system may be used to determine air contamination, composition of liquid, effectiveness of medication on microorganisms et cetera.

While the invention has been illustrated and described in detail in the drawings and the foregoing description, such illustration and description are to be considered illustrative or exemplary and not restrictive.

From reading the present disclosure, other modifications will be apparent to persons skilled in the art. Such modifications may involve other features which are already known in the art and which may be used instead of or in addition to features already described herein.

Variations to the disclosed embodiments can be understood and effected by those skilled in the art, from a study of the drawings, the disclosure and the appended claims. In the claims, the word “comprising” does not exclude other elements or steps, and the indefinite article “a” or “an” does not exclude a plurality of elements or steps. The mere fact that certain measures are recited in mutually different dependent claims does not indicate that a combination of these measures cannot be used to advantage.

Any reference signs in the claims should not be construed as limiting the scope thereof.

### LIST OF REFERENCE NUMERALS

101 first fluid channel unit
102 second fluid channel unit
103 third fluid channel unit
104 fourth fluid channel unit
105 fifth fluid channel unit
106 sixth fluid channel unit
111 first fluid inlet
112 second fluid inlet
115 socket
121 first fluid outlet
122 second fluid outlet
131 first fluid treatment unit
132 second fluid treatment unit
133 third fluid treatment unit
134 fourth fluid treatment unit
135 fifth fluid treatment unit
150 fluid sensing terminal
151 fluid outlet of the terminal
152 evaluator
154 terminal data interface
156 user interface
157 movable wall
158 separator
159 fluid pump controller
161 first fluid pump
162 second fluid pump
171 first sensor
172 second sensor
173 third sensor
174 fourth sensor
181 dummy unit
191 first seal
192 second seal
193 third seal
200 sensor module
250 air purifier
310 step of measuring TVOC
320 step of removing (Form)aldehydes
330 step of measuring TVOC—(Form)aldehydes
340 step of removing other VOC
350 step of measuring remaining VOC concentration

1. A fluid channel unit comprising at least a first fluid inlet, a first fluid outlet and at least a second fluid outlet, wherein the fluid channel unit is arranged such that the fluid can flow from the first fluid inlet to the first fluid outlet and from the first fluid inlet to the second fluid outlet, wherein the fluid channel unit is further removably coupled to a fluid sensing terminal and at least a second fluid channel unit and a third fluid channel unit such that the fluid can flow from the first fluid outlet of the fluid channel unit to the first fluid inlet of the second fluid channel unit and such that the fluid can flow from the second fluid outlet of the fluid channel unit to the first fluid inlet of the third fluid channel unit,
wherein:
the fluid channel unit further comprising at least one unit identification interface, wherein the unit identification interface is adapted to exchange configuration information regarding a fluid treatment unit, sensor or seal coupled to the fluid channel unit with the fluid sensing terminal.

2. The fluid channel unit according to claim 1 comprising a casing, wherein the casing is adapted to align the fluid channel unit to the fluid sensing terminal.

3. The fluid channel unit according to claim 1 comprising at least one socket arranged at the first fluid inlet, first fluid outlet or second fluid outlet, wherein the socket is arranged to receive at least one device selected out of the group of fluid treatment unit, sensor or seal.

4. The fluid channel unit according to claim 1 comprising at least one sensor, wherein the sensor is arranged in a channel connecting the first fluid inlet and the first fluid outlet, or wherein the sensor is arranged in a channel connecting the first fluid inlet and the second fluid outlet, and wherein the fluid channel unit further comprises a sensor data interface, wherein the sensor data interface is adapted to exchange a measurement result of the sensor with the fluid sensing terminal.

5. The fluid channel unit according to claim 1 comprising at least one fluid treatment unit, wherein the fluid treatment unit is arranged in a channel connecting the first fluid inlet and the first fluid outlet, or wherein the fluid treatment unit is arranged in a channel connecting the first fluid inlet and the second fluid outlet.

6. The fluid channel unit according to claim 1 comprising at least one fluid pump for moving the fluid in or out of the fluid channel unit, wherein the fluid pump is arranged in a channel connecting the first fluid inlet and the first fluid outlet, or wherein the fluid pump is arranged in a channel connecting the first fluid inlet and the second fluid outlet, and wherein the fluid channel unit further comprises an electrical interface being adapted to supply electrical power to the fluid pump from the fluid sensing terminal.

7. The fluid channel unit according to claim 1, wherein the fluid channel unit comprises at least a second fluid inlet.

8. A fluid sensing terminal comprising:
a coupling opening for placing and removably coupling at least three fluid channel units according to claim 1, wherein the at least three fluid channel units can be removably coupled to the fluid sensing terminal such that that the fluid can flow from at least one fluid inlet of the fluid sensing terminal via a first fluid inlet and a first fluid outlet of a first fluid channel unit to the first fluid inlet of the second fluid channel unit, and such that the fluid can flow from the fluid inlet of the fluid sensing terminal via the first fluid inlet and a second fluid outlet of the first fluid channel unit to the first fluid inlet of the third fluid channel unit,
wherein the fluid sensing terminal further comprises an evaluator, and wherein the evaluator is adapted to receive at least four sensor signals resulting from fluid property measurements of a fluid passing a first fluid outlet of the second fluid channel unit, a fluid passing a second fluid outlet of the second fluid channel unit, a fluid passing a first fluid outlet of the third fluid channel unit and a fluid passing a second fluid outlet of the third fluid channel unit, and
wherein the fluid sensing terminal comprises at least one of a user interface for presenting at least a part of results of the fluid property measurements to a user of the fluid sensing terminal or a terminal data interface for exchanging data comprising at least a part of the results of the fluid property measurements, and
wherein the fluid sensing terminal further comprises at least one first sensor, wherein the first sensor is adapted to measure at least the fluid property at least of the fluid passing the first fluid outlet of the second fluid channel unit, the fluid passing the second fluid outlet of the second fluid channel unit, the fluid passing the first fluid outlet of the third fluid channel unit and the fluid passing the second fluid outlet of the third fluid channel unit.

9. The fluid sensing terminal according to claim 8 further comprising at least one first fluid pump for moving the fluid via the at least one fluid inlet of the fluid sensing terminal to at least one fluid outlet of the fluid sensing terminal.

10. The fluid sensing terminal according to claim 8, wherein the evaluator is adapted to receive identification information from at least one device selected out of the group fluid channel unit, fluid treatment unit, sensor or seal.

11. The fluid sensing terminal according to claim 10, wherein the fluid sensing terminal comprises at least one terminal identification interface for each fluid channel unit, wherein the terminal identification interfaces are adapted to receive the identification information via a corresponding unit identification interface of the fluid channel units.

12. The fluid sensing terminal according to claim 10, wherein the evaluator is adapted to perform a configuration detection procedure for determining an arrangement of the fluid channel units in the fluid sensing terminal after coupling the fluid channel units with the fluid sensing terminal.

13. A fluid sensing system comprising at least one fluid sensing terminal according to claim 8 and at least three fluid channel units.
